(19) United States
(12) Patent Application Publication
(10) Pub. No.: US 2017/0215804 A1
(43) Pub. Date: Aug. 3, 2017

(54) OBJECT INFORMATION ACQUIRING APPARATUS AND SIGNAL PROCESSING METHOD

(71) Applicant: CANON KABUSHIKI KAISHA, Tokyo (JP)

(72) Inventor: Takuro Miyasato, Tokyo (JP)

(21) Appl. No.: 15/405,392

(22) Filed: Jan. 13, 2017

(30) Foreign Application Priority Data
Jan. 29, 2016 (JP) 2016-015107

(51) Int. Cl. A61B 5/00; A61B 5/1455

(52) U.S. Cl. CPC A61B 5/7203; A61B 5/0095; A61B 5/14551; A61B 2562/0238; A61B 2576/00

(57) ABSTRACT

Disclosed is an object information acquiring apparatus including: an irradiator configured to emit pulsed light of a plurality of wavelengths different from each other; a receiver configured to receive object signals propagating from the object irradiated with the pulsed light of the plurality of wavelengths and converts the received object signals into a plurality of reception signals; a corrector configured to correct at least any of the plurality of reception signals according to pulse shapes of beams of the pulsed light of the plurality of wavelengths; and an information acquirer configured to acquire spectral information on the object using the plurality of reception signals corrected by the corrector.

### BACKGROUND OF THE INVENTION

Field of the Invention

The present invention relates to an object information acquiring apparatus and a signal processing method.

Description of the Related Art

Research on an apparatus that causes pulsed light of a plurality of wavelengths, which is irradiated from a light source such as a laser to an object, to propagate through the inside of the object to acquire information on the inside of the object has been actively promoted mainly in the field of medicine. As one of spectral information acquiring technologies using such pulsed light, U.S. Pat. No. 5,713,356 discloses photoacoustic tomography (hereinafter referred to as PAT). In addition, U.S. Pat. No. 8,000,775 proposes diffuse optical tomography (hereinafter referred to as DOT). In either of the technologies, it is possible to calculate oxygen saturation, component concentration, function information, or the like inside an object by the irradiation of pulsed light of a plurality of wavelengths. However, in order to accurately calculate such information, it is necessary to irradiate the object with delta type short pulsed light such that its pulse shape becomes the same between the plurality of wavelengths.

Patent Literature 1: U.S. Pat. No. 5,713,356

Patent Literature 2: U.S. Pat. No. 8,000,775

### SUMMARY OF THE INVENTION

However, in some cases, it is hard to emit delta type short pulsed light such that its pulse shape becomes the same between a plurality of wavelengths. For this reason, the pulse shape is different between the respective wavelengths, and accuracy in calculating component concentration, oxygen saturation, or function information using the ratio between the wavelengths deteriorates.

The present invention has been made in view of the above problems. The present invention has an object of improving accuracy in acquiring information in an apparatus that irradiates an object with light of a plurality of wavelengths to acquire information inside the object.

The present invention provides an object information acquiring apparatus comprising:

an irradiator configured to emit pulsed light of a plurality of wavelengths different from each other;

a receiver configured to receive object signals propagating from the object irradiated with the pulsed light of the plurality of wavelengths and convert the received object signals into a plurality of reception signals;

a corrector configured to correct at least any one of the plurality of reception signals according to pulse shapes of the pulsed light of the plurality of wavelengths; and

an information acquirer configured to acquire spectral information on the object using the plurality of reception signals corrected by the corrector.

The present invention provides a signal processing method for a plurality of reception signals acquired by receiving object signals propagating from an object irradiated with pulsed light of a plurality of wavelengths different from each other,

the method comprising:

correcting at least any of the plurality of reception signals according to pulse shapes of beams of the pulsed light of the plurality of wavelengths; and

acquiring spectral information on the object using the plurality of reception signals corrected in the correction step.

According to an embodiment of the present invention, it is possible to improve accuracy in acquiring information in an apparatus that irradiates an object with light of a plurality of wavelengths to acquire information inside the object.

Further features of the present invention will become apparent from the following description of exemplary embodiments with reference to the attached drawings.

### BRIEF DESCRIPTION OF THE DRAWINGS

FIG. 1 is an apparatus diagram for describing an embodiment of the present invention;

FIG. 2 is a flowchart for describing the embodiment of the present invention;

FIG. 3 is an apparatus diagram for describing a first embodiment;

FIG. 4 is a flowchart for describing the first embodiment;

FIG. 5 is an apparatus diagram for describing a second embodiment;

FIG. 6 is an apparatus diagram for describing a third embodiment; and

FIG. 7 is a flowchart for describing a third embodiment.

### DESCRIPTION OF THE EMBODIMENTS

Hereinafter, a description will be given of the preferred embodiments of the present invention with reference to the drawings. However, sizes, materials, shapes, their relative arrangements, or the like of constituents that will be described below may be appropriately changed depending on the configurations or various conditions of apparatuses to which the present invention is applied. Accordingly, the sizes, materials, shapes, their relative arrangements, or the like of the constituents do not intend to limit the scope of the invention to the following descriptions.

The present invention relates to a technology for detecting acoustic waves propagating from an object and generating and acquiring characteristic information inside the object. Accordingly, the present invention is grasped as an object information acquiring apparatus or its control method, or grasped as an object information acquiring method or a signal processing method. In addition, the present invention is grasped as a program that causes an information processing apparatus having hardware resources such as a CPU and a memory to perform these methods, or grasped as a storage medium storing the program.

The object information acquiring apparatus of the present invention includes a photoacoustic imaging apparatus using a photoacoustic effect in which acoustic waves generated inside an object by irradiating the object with light (electromagnetic waves) are received to acquire characteristic information on the object as image data. In this case, the characteristic information indicates information on characteristic values corresponding to a plurality of positions inside the object, the information being generated using reception signals obtained by the reception of photoacoustic waves.

Characteristic information acquired by photoacoustic measurement is a value reflecting the absorption ratio of light energy. For example, the characteristic information includes the generation source of acoustic waves generated by the irradiation of light of a single wavelength, initial sound pressure inside an object, or light energy absorption density or an absorption coefficient derived from initial sound pressure. In addition, information acquired from characteristic information acquired by a plurality of wavelengths different from each other is called spectral information. A typical example of the spectral information includes substance concentration constituting tissues. It is possible to calculate an oxygen saturation distribution by the calculation of oxyhemoglobin concentration and deoxyhemoglobin concentration as the substance concentration. In addition, glucose concentration, collagen concentration, melanin concentration, a volume fraction of fat or water, or the like is calculated as the substance concentration.

Based on characteristic information on respective positions inside an object, a two-dimensional or three-dimensional characteristic information distribution is acquired. Distribution data may be generated as image data. The characteristic information may be calculated as distribution information on respective positions inside the object rather than being calculated as numerical data. That is, the characteristic information is distribution information such as an initial sound pressure distribution, an energy absorption density distribution, an absorption coefficient distribution, and an oxygen saturation distribution.

In the present invention, acoustic waves are typically ultrasonic waves and include elastic waves called sound waves or acoustic waves. Electrical signals converted from the acoustic waves by a transducer or the like are also called acoustic signals. However, in the specification, the ultrasonic waves or acoustic waves do not intend to limit the wavelengths of such elastic waves. The acoustic waves generated by the photoacoustic effect are called photoacoustic waves or light ultrasonic waves. Electrical signals derived from the photoacoustic waves are also called photoacoustic signals.

As a type of the photoacoustic imaging apparatus according to the present invention, a photoacoustic microscope has been known in which sound is focused or pulsed light is converged to increase the resolution of photoacoustic imaging. According to the photoacoustic microscope, it is possible to increase resolution to image finer light absorbers.

The object information acquiring apparatus of the present invention also includes an apparatus that detects light propagating through the inside of an object after the irradiation of the object and calculates an optical characteristic value distribution inside the object from the intensity of the light. In this case, object information is function information such as an average optical coefficient, an absorption coefficient, a scattering coefficient, and oxygen saturation inside the object. A technology for acquiring such optical characteristic values and generating image data inside an object from the optical characteristic values is called diffuse optical tomography (DOT).

That is, the object information acquiring apparatus of the present invention irradiates an object with pulsed light and analyzes signals output from the object to acquire spectral information on the inside of the object. Further, the object information acquiring apparatus is an apparatus that acquires a substance concentration distribution, oxygen saturation, function information, or the like as spectral information. Accordingly, it may also be said that the present invention targets at a spectral information acquiring apparatus or a spectral information acquiring method. The signals output from the object may be light of the same wavelength as that of irradiated pulsed light or light of different wavelengths. Further, the signals may be acoustic signals acquired by the photoacoustic effect. The present invention is particularly preferably applied to a spectral information acquiring apparatus using light of a plurality of wavelengths. For example, the present invention may also target at a near-infrared light imaging apparatus using light of a plurality of wavelengths or the like.

### Embodiment of Present Invention

(Configuration Outline of Apparatus)

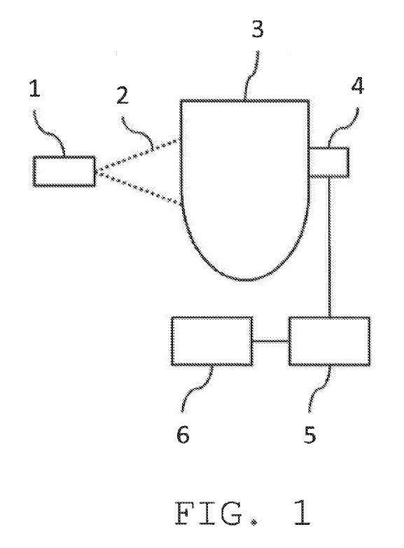

FIG. 1 is a schematic diagram of a spectral information acquiring apparatus according to the embodiment. The spectral information acquiring apparatus according to the embodiment includes a light irradiator 1 that irradiates an object with pulsed light of at least two or more types of wavelengths, a receiver 4, a pulse shape difference corrector 5, and a spectral information acquirer 6. An object 3 is a measurement target, and a light absorption coefficient distribution or a light scattering coefficient distribution exists inside the object 3. When a living body is used as an object in photoacoustic imaging, hemoglobin contained in living body tissues, blood vessels containing the hemoglobin, tumors including many new blood vessels, or the like are preferable as light absorbers.

The irradiator 1 is composed of a light source that emits pulsed light and an irradiation optical system that irradiates an object with the emitted pulsed light. The object 3 is irradiated with the pulsed light serving as irradiation pulsed light 2. The irradiation pulsed light 2 entering the object is diffused and absorbed in the object. As a result, generated signals propagate through the inside of the object, are detected by the receiver 4, and are converted into analog electrical signals. The electrical signals are further subjected to amplification or digital conversion and then stored in a memory (not shown) as reception data.

The pulse shape difference corrector 5 (corrector) performs a correction to absorb a difference caused by the pulse shape difference between reception data of respective wavelengths according to the impulse responses of the receiver considering the pulse shapes or the pulse widths of the respective wavelengths measured in advance. The pulse shapes indicate the time intensity changes of light pulses, i.e., changes in the intensity of the light pulse with time. In addition, the spectral information acquirer 6 (information acquirer) calculates the absorption coefficients or the scattering coefficients of respective wavelengths using the corrected reception data and calculates spectral information from the ratio between the wavelengths.

(Irradiator)

The irradiator 1 is composed of the light source and the irradiation optical system. The light source is preferably capable of generating pulsed light of 5 to 50 nanoseconds. The light source is preferably a laser capable of producing a large output. However, a light-emission diode, a flash lamp, or the like may be used. The laser may include various lasers such as a solid-state laser, a gas laser, a dye laser, and a semiconductor laser. Ideally, a Ti:Sa laser pumped by a Nd:YAG laser, an alexandrite laser, or the like that produces a large output and is capable of continuously changing wavelengths may be used. A plurality of short-wavelength lasers having different wavelengths may be used.

An object is irradiated with the pulsed light emitted from the light source via the irradiation optical system. The irradiation optical system includes optical components such as a mirror that reflects light, a lens that expands the light, and a diffusion plate that diffuses the light. The irradiation optical system guides the pulsed light into the object while forming the same into a desired irradiation light distribution shape. The irradiation optical system also includes a waveguide such as an optical fiber that causes light to propagate. Any irradiation optical system may be used so long as the object 3 is desirably irradiated with the pulsed light emitted from the light source. Note that it is more preferable to widen light so as to have an area of a certain degree rather than converging the same with a lens in terms of safety for the object and an increase in a diagnosis area. In order to irradiate a wider area of the object with the pulsed light, a light irradiator scanning mechanism may be provided in the light irradiation optical system. In addition, the irradiation optical system may have a plurality of light emission ports that allows the selection of an irradiation position.

(Receiver)

The receiver 4 has a reception device that physically receives object signals and a signal processing mechanism. In the case of a photoacoustic apparatus, the object signals are photoacoustic waves generated on the front surface and the inside of an object, and the receiver is an acoustic wave reception device that detects acoustic waves and converts the same into electrical signals serving as analog signals. As the acoustic wave reception device, it is possible to use, for example, a transducer using a photoelectric phenomenon, a transducer using the resonance of light, a transducer using a change in capacity, or the like.

Note that in order to detect photoacoustic waves at a plurality of positions, it is preferable to use an array probe in which a plurality of photoacoustic receiving elements are one-dimensionally or two-dimensionally arranged side by side or a three-dimensional probe in which a plurality of acoustic wave detecting elements is arranged on the inner peripheral surface of a hemispherical container. In addition, it is preferable to provide a mechanical scanning mechanism in order to change the relative position between the receiver and the object. Since these configurations allow the measurement of the object in a wider range, an improvement in measurement accuracy, an increase in SN ratio, a reduction in measurement time, or the like is expected. In addition, a single element focused by an acoustic lens may be used to specify the position of an acoustic wave generation source.

On the other hand, object signals in an apparatus using diffused light or the like are light propagating from an object and having wavelengths the same as or different from that of irradiation pulsed light irradiated by an irradiator. The receiver is a light detection device that detects light serving as object signals and converts the same into electrical signals serving as analog signals. As such, a photomultiplier or a semiconductor photodiode using a photoelectric effect, a pyroelectric detection device using a thermal effect, a Golay cell, a bolometer, or the like may be, for example, used.

The signal processing mechanism amplifies electrical signals obtained by the reception device and converts the electrical signals from analog signals into digital signals. The signal processing mechanism is typically composed of an amplification device, an A/D conversion device, a field programmable gate array (FPGA) chip, or the like. When a plurality of detection signals is obtained from an acoustic wave reception device or a light reception device, the signal processing mechanism preferably processes the plurality of signals at the same time to shorten a processing time. In addition, reception signals received at the same position of an object may be integrated into one signal. As an integration method, the simple integration of signals, the acquisition of an average of signals, the adding up of weighted signals, or the like may be used. Note that the “reception signals” in the specification are concepts including both analog signals output from the acoustic reception device or the light detection device and digital signals obtained by subjecting the analog signals to AD conversion.

(Pulse Shape Difference Corrector)

In the embodiment, a time-series reception signal is obtained for each of the wavelengths of pulsed light. From the plurality of reception signals, the pulse shape difference corrector 5 performs a correction so as to reduce influence caused by the pulse shape difference between the wavelengths to acquire corrected reception signals. That is, the pulse shape difference corrector 5 performs a correction so as to reduce the difference between the pulse widths of a plurality of reception signals to acquire corrected reception signals. The pulse shape difference corrector may be included in the signal processing mechanism as a program or an FPGA chip or may be included as a program in a workstation serving as the spectral information acquirer that will be described later. Any correction method may be used so long as a correction to reduce influence caused by the pulse shape difference between the wavelengths of reception signals is made possible.

(Method for Correcting Signals)

Next, a description will be given of a method for correcting the pulse shape difference between the wavelengths of reception signals performed by the pulse shape difference corrector. When measurement is performed using N wavelengths (wavelengths 1 to n), it is assumed that the pulse shapes of the respective wavelengths are indicated as P1(t) to Pn(t). Here, t indicates a time. It is preferable to measure the light pulse shapes of the respective wavelengths with an oscilloscope or the like in advance. Even if the pulse shapes are not completely measurable, it suffices if only pulse widths are measured. A pulse typically includes but not limited to delta type short pulsed light. In addition, reception signals obtained when an object is measured using light beams of the respective wavelengths are indicated as S1(t) to Sn(t). Five correction methods will be described below.

(First Correction Method) First, a description will be given of a method for correcting the reception signal of another wavelength relative to the pulse shape of one reference wavelength. When it is assumed that the reference wavelength is placed in the k-th order, the pulse shape of the reference wavelength is indicated as Pk(t) and a signal derived from the pulsed light of the reference wavelength is indicated as Sk(t). The reference wavelength is not particularly limited. However, it is preferable to select, as the reference wavelength, a wavelength having the largest pulse width to prevent noise or the like from increasing when the correction is performed.

Here, a description will be given of a method for correcting the n-th (where n≠k) reception signal. It is assumed that the n-th pulse shape is indicated as Pn(t) and values obtained by subjecting the k-th pulse shape and the n-th pulse shape to Fourier transform are indicated as FT(Pk(t)) and FT(Pn(t)), respectively. A function FT(x) is a function used to subject x to Fourier transform. In addition, a value obtained by subjecting a reception signal Sn(t) to Fourier transform is indicated as FT(Sn(t)). When the signal Sn(t) is corrected using the pulse shape Pk(t) of the reference k-th wavelength and the pulse shape Pn(f) of the n-th wavelength, corrected reception signals are acquired by calculation using a frequency domain as shown in the following Formula (1).

$$\begin{matrix}[ Math . 1 ] & \\ corr 1 ( Sn ( t ) ) = i FT [ \frac{\overline{FT ( Pk ( t ) )}}{{FT ( Pn ( t ) )}^{2} + {SN}^{2}} \cdot FT \{ S n ( t ) \} ] & ( 1 )\end{matrix}$$

where \overline{FT{Pk(y)}} indicates the complex conjugate of FT{Pk(t)}.

SN indicates a threshold for noise and serves as a term for reducing excessive noise or zero divide. In addition, iFT[x] indicates a function for subjecting x to reverse Fourier transform. By the application of the above Formula (1) to the reception signals S1(t) to Sn(t) other than the k-th reception signal, corr1(S1(t)) to corr1(Sn(t)) are acquired.

(Second Correction Method) Second, a description will be given of another method for correcting the reception signal of another wavelength relative to the pulse width of a reference wavelength. When it is assumed that the reference wavelength is placed in the k-th order, the pulse shape of the reference wavelength is indicated as Pk(t). It is assumed that a value obtained by approximating the pulse shape with a Gaussian function is indicated as G(Pk(t)) and its half-value width is indicated as HWG(Pk(t)). It is assumed that the half-value width HWG(Pk(t)) is the pulse width of the k-th wavelength. Here, the pulse shape is approximated with the Gaussian function, but any function may be used so long as the approximation of the pulse shape Pk(t) is made possible.

In addition, it is most preferable that the reference wavelength have the largest pulse width. This is because, when the signal of a wavelength having a shorter pulse width than that of the reference wavelength is performed, it is only necessary to perform blurring or the convolution of the Gaussian function. On the other hand, when the signal of a wavelength having a longer pulse width than that of the reference wavelength is corrected, it is necessary to perform deconvolution, whereby it is highly likely that noise increases. As for other wavelengths, it is assumed that values obtained by approximating pulse shapes with the Gaussian function are indicated as G(P1(t)) to G(Pn(t)) and their half-value widths are indicated as HWG(P1(t)) to HWG(Pn(t)). From the approximated pulse widths of the respective wavelengths, correction pulse widths are calculated by the following Formula (2).

[Math. 2]

cHWG(Pn(t))={square root over ((HWG(Pk(t))²−HWG(Pn(t))²))}(HWG(Pk(t))≧HWG(Pn(t)))

cHWG(Pn(t))={square root over ((HWG(Pk(t))²−HWG(Pn(t))²))}(HWG(Pk(t))<HWG(Pn(t))) (2)

When it is assumed that the normal distribution function of the central value 0 is indicated as NDF(cHWG(Pn(t))) at a half-value width cHWG(Pn(t)), the pulse shape difference between the wavelengths of the reception signals is corrected by the following Formula (3).

[Math. 3]

corr2(Sn(t))=iFT[FT{Sn(t)}·FT{NDF(cHWG(Pn(t)))}](HWG(Pk(t))≧HWG(Pn(t)))

corr2(Sn(t))=iFT[FT{Sn(t)}/FT{NDF(cHWG(Pn(t)))}](HWG(Pk(t))≧HWG(Pn(t))) (3)

(Third Correction Method) Third, a description will be given of a method for eliminating the influence of the pulse shapes of respective wavelengths from all signals. It is assumed that the corrected signals of the respective wavelengths are indicated as corr3(S1(t)) to corr3(Sn(t)). By the following Formula (4), the pulse shape difference between the wavelengths of the reception signals is corrected at the respective wavelengths.

[Math. 4]

corr3(Sn(t))=iFT[FT{Sn(t)}/FT(Pn(t))] (4)

(Fourth Correction Method) Fourth, a description will be given of another method for eliminating the influence of the pulse widths of respective wavelengths from all signals. It is assumed that values obtained by approximating the pulse shapes of the respective wavelengths with a Gaussian function are indicated as G(P1(t)) to G(Pn(t)). In addition, the corrected signals of the respective wavelengths are indicated as corr4(S1(t)) to corr4(Sn(t)). By the following Formula (5), the pulse width difference between the wavelengths of the reception signals is corrected at the respective wavelengths.

[Math. 5]

corr4(Sn(t))=iFT[FT(Sn(t))/FT{G(Pn(t))}] (5)

(Fifth Correction Method) Fifth, a description will be given of a method for measuring in advance the impulse responses of a detection device with consideration given to the influence of pulse shapes at respective wavelengths and correcting the reception signals of the respective wavelengths using the impulse responses. It is assumed that the impulse responses of the detection device with consideration given to the influence of the pulse shapes of the respective wavelengths are indicated as IR1(t) to IRn(t) and the corrected signals of the respective wavelengths are indicated as corr5(S1(t)) to corr5(Sn(t)). By the following Formula (6), the impulse responses and the pulse shapes of the reception signals are corrected at the respective wavelengths. The impulse responses are preferably measured in advance and stored in a memory in the form of a table or a mathematical formula.

[Math. 6]

corr5(Sn(t))=iFT[FT{Sn(t)}/FT{IRn(t)}] (6)

(Spectral Information Acquirer)

Referring back to FIG. 1, the description will be continued. The spectral information acquirer 6 performs the calculation of a light amount distribution, image reconfiguration, or the like to acquire spectral information inside the object. As the spectral information acquirer, a PC or a workstation including a calculation resource such as a CPU and a memory is preferably used. Image reconfiguration processing is performed according to software programmed in advance.

The spectral information acquirer 6 calculates an initial sound pressure distribution P0(r) using reception signals corrected by the pulse shape difference corrector. The initial sound pressure distribution may be calculated according to a universal back projection (UBP) method, phase addition (delay and sum), a repeat inverse problem method, or model-based reconfiguration. Any method may be used unless the quantitativeness between wavelengths is impaired.

The spectral information acquirer 6 further calculates a light amount distribution φ(r) inside the object when the object is irradiated with light beams of the respective wavelengths, and divides the initial sound pressure distribution P0(r) by the calculated light amount distribution φ(r) and a Gruneisen coefficient to calculate the distribution of an absorption coefficient distribution μa(r) at the respective wavelengths. In addition, a component concentration distribution or an oxygen saturation distribution is calculated using the absorption coefficient distribution of the respective wavelengths.

<Processing Procedure>

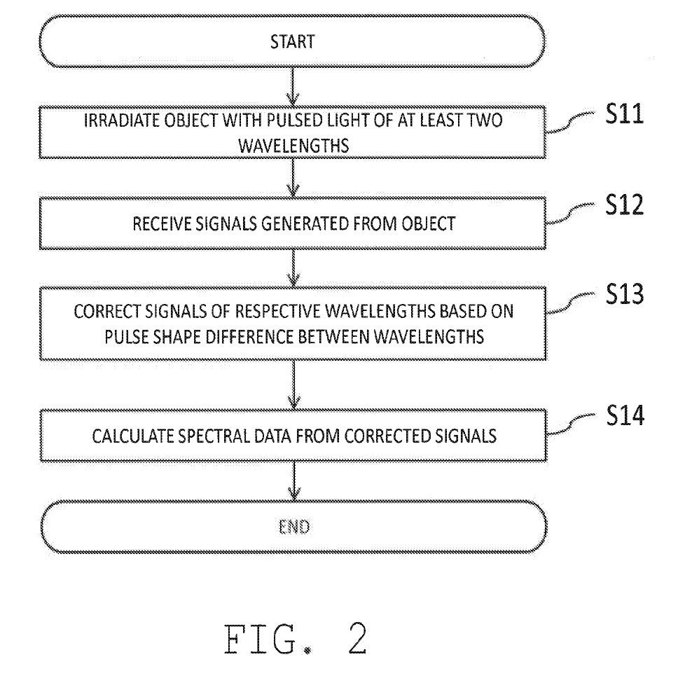

A description will be given of the spectral information acquiring method of the embodiment with reference to the flowchart of FIG. 2.

(Step S11: Irradiation Step)

The light irradiator 1 irradiates an object with pulsed light of at least two or more wavelengths.

(Step S12: Signal Reception Step)

As the object is irradiated with the pulsed light in step S11, the receiver 4 receives signals (light or acoustic waves) emitted from the object. The light or the acoustic waves are converted into electrical signals, and then stored in a memory or the like.

(Step S13: Signal Correction Step)

The pulse shape difference corrector 5 corrects the reception signals of the respective wavelengths received in step S12 according to the pulse shape difference between the respective wavelengths to acquire corrected reception signals.

(Step S14: Spectral Data Calculation Step)

The spectral information acquirer 6 calculates characteristic information such as an initial sound pressure distribution and an absorption coefficient distribution at the respective wavelengths using the corrected reception signals calculated in step S13. In addition, depending on a processing content, the spectral information acquirer 6 calculates final spectral information (such as an oxygen saturation distribution) using characteristic information at the plurality of wavelengths.

According to the spectral information acquiring method of the above flow, the corrected reception signals are acquired in which the pulse shape difference between the plurality of wavelengths is corrected in step S13. As a result, the accuracy or the resolution of image data generated in step S14 is improved.

### First Embodiment

Subsequently, a description will be given of a more specific embodiment. First, in a first embodiment, a description will be given of a PAT diagnosis apparatus that measures a human breast with a bowl-shaped probe to acquire blood vessel information inside the breast or the oxygen saturation distribution of blood. A signal correction in the first embodiment is correction processing based on a pulse width difference.

(Configuration)

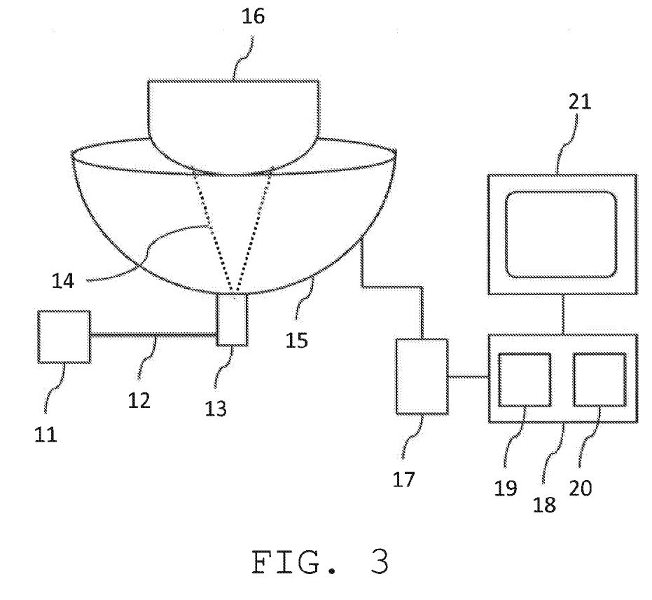

A description will be give of the configuration of the embodiment with reference to FIG. 3. An alexandrite laser 11 emits pulsed light of wavelengths of 756 nm and 797 nm. The pulsed light enters an irradiation optical system 13 via an articulated arm 12. Then, the pulsed light is magnified via a mirror, a lens, and a diffusion plate. After that, a human breast 16 serving as an object is irradiated with the pulsed light serving as pulsed light 14. The alexandrite laser 11 corresponds to the light source of an irradiator.

The bowl-shaped probe 15 is filled with water, and the breast 16 is immersed in the water. Acoustic waves emitted from the breast 16 by a photoacoustic effect are received by a plurality of piezo elements arranged on the bowl-shaped probe 15 in a Fibonacci arrangement pattern, and then converted into electrical signals. The electrical signals are subjected to amplification processing and digital conversion processing, and then stored in a memory (not shown) inside a data acquiring system 17. The bowl-shaped probe 15 corresponds to a receiver.

The reception signals derived from the respective wavelengths stored in the memory inside the data acquiring system 17 are converted into reception signals in which the pulse width difference between the wavelengths is corrected (hereinafter, inter-wavelength pulse width difference correction signals) according to a prescribed program in a mechanism 19 that corrects the pulse width difference between the wavelengths (hereinafter, an inter-wavelength pulse width difference correction mechanism 19) constituting a PC 18. The inter-wavelength pulse width difference correction mechanism 19 acquires pulse width data on the wavelengths of 756 nm and 797 nm measured in advance from the memory, an external apparatus, or the like, and uses the acquired pulse width data to perform a correction. The inter-wavelength pulse width correction mechanism 19 corresponds to a pulse shape difference corrector.

The oxygen saturation calculation mechanism 20 calculates a hemoglobin concentration distribution and an oxygen saturation distribution using the inter-wavelength pulse width difference correction signals. The PC 18 causes an image, in which the hemoglobin concentration distribution and the oxygen saturation distribution are allocated to brightness and hue, respectively, to be displayed on a display 21. Here, the hemoglobin concentration distribution is allocated to brightness but needs only to be allocated to any of brightness, chroma, and hue. The oxygen saturation calculation mechanism 20 corresponds to a spectral information acquirer.

(Correction Method)

A description will be given in detail of the inter-wavelength pulse width difference correction of the embodiment. Light of a wavelength of 756 nm has a pulse width of 60 nsec, and light of a wavelength of 797 nm has a pulse width of 90 nsec. Here, the pulse width indicates the half-value width of a Gaussian function obtained by fitting the pulse shapes of the respective wavelengths with the Gaussian function. Here, it is expected to perform a correction as if a reception signal S₇₅₆(t) derived from the light of a wavelength of 756 nm was obtained by the irradiation of the pulsed light of a pulse width of 90 nsec. To this end, it is only necessary to perform blurring with the Gaussian distribution of the half-width value √(90̂2−60̂2)=67 nsec.

Note that it is also possible to perform a correction as if a reception signal S₇₉₇(t) derived from the light of a wavelength of 797 nm was obtained by the irradiation of the pulsed light of a pulse width of 60 nsec. In this case, deconvolution with the Gaussian function of the half-value width of the half-value width 67 nsec is performed. However, it is necessary to reduce an increase in noise due to the deconvolution as soon as possible. Therefore, when a higher value is placed on quantitativeness than resolution to acquire spectral information, it is preferable to adjust a reception signal of a wavelength of a smaller pulse width to a reception signal of a wavelength of a larger pulse width. Then, by masking the resulting spectral information with characteristic information (for example, an initial sound pressure distribution or an absorption coefficient distribution) obtained by the wavelength of a smaller pulse width, it is possible to acquire spectral information assuring both quantitativeness and resolution. For example, the masking may be performed in such a way that the value of the characteristic information obtained at the wavelength of a smaller pulse width is allocated to any of hue, chroma, and brightness of the spectral information as described above.

(Processing Flow)

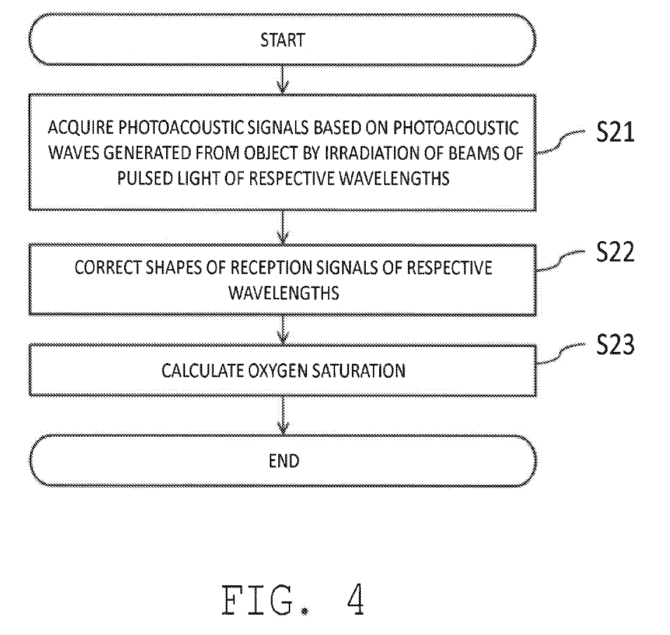

A description will be given of the spectral information acquiring method of the embodiment with reference to the flowchart of FIG. 4.

(Step S21)

The breast is irradiated with pulsed light of two wavelengths to acquire photoacoustic signals. That is, the breast 16 is irradiated with the beams of the pulsed light 14 of wavelengths of 756 nm and 797 nm by the irradiation optical system 13. The piezo elements arranged side by side on the bowl-shaped probe 15 receive photoacoustic waves derived from the light beams of the respective wavelengths and convert the same into electrical signals. After the conversion, the piezo elements store the electrical signals in the data acquiring system 17.

(Step S22)

The reception signals of the respective wavelengths are subjected to a pulse width difference correction according to the pulse widths of the respective wavelengths. That is, the inter-wavelength pulse width difference correction mechanism 19 generates corrected reception signals based on the pulse widths of the pulsed light of wavelengths of 756 nm and 797 nm. Here, the reception signal of a wavelength of 756 nm is adjusted to the reception signal of a wavelength of 797 nm of a relatively larger pulse width.

(Step S23)

An absorption coefficient distribution is calculated by the pulse width difference correction reception signals, and an oxygen saturation distribution is also calculated. That is, the oxygen saturation calculation mechanism 20 calculates an initial sound pressure distribution using a UPB method based on the corrected reception signal derived from the light of a wavelength of 756 nm and the reception signal derived from the light of a wavelength of 797 nm. Specifically, according to the formula (μa=P0/┌φ), the initial sound pressure distribution is divided by a light amount distribution φ and a Gruneisen coefficient to calculate an absorption coefficient. Then, a hemoglobin concentration distribution and an oxygen saturation distribution are calculated from the absorption coefficient distribution of the light of wavelengths of 756 nm and 797 nm. The PC 18 allocates the hemoglobin concentration distribution and the oxygen saturation distribution to brightness and hue, respectively, and causes the same to be displayed on the display 21 as a hemoglobin oxygen saturation distribution.

According to the processing with the spectral information acquiring apparatus of the embodiment, it is possible to correct the pulse width difference between wavelengths in a photoacoustic apparatus that acquires substance concentration and oxygen saturation using light of a plurality of wavelengths. As a result, it is possible to accurately image a characteristic information distribution inside an object.

### Second Embodiment

A second embodiment will describe an apparatus that measures the brain of a mouse with a light focus type photoacoustic microscope and observes a change in the blood amount or the oxygen saturation of the brain of the mouse due to a stimulus.

(Configuration)

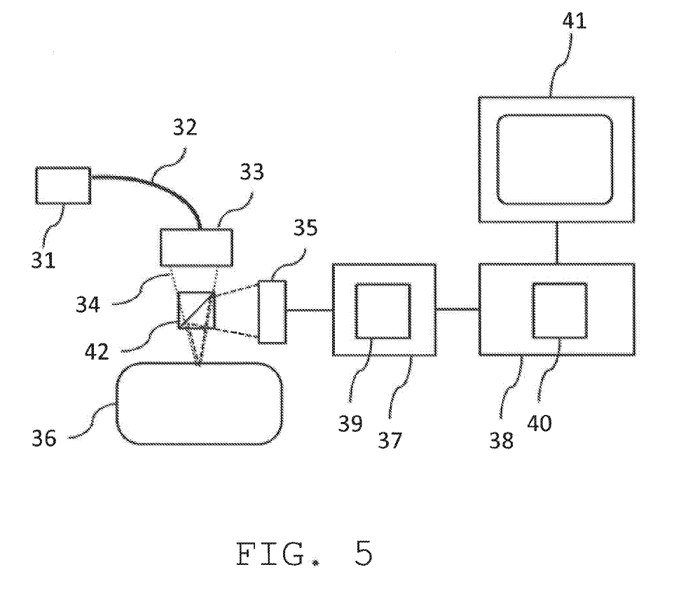

A description will be given of the configuration of the embodiment with reference to FIG. 5. A Ti:sa laser 31 is capable of emitting pulsed light of four wavelengths of 756 nm, 780 nm, 797 nm, and 825 nm. The emitted pulsed light enters an irradiation optical system 33 via a light bundle fiber 32. The pulsed light passes through a mirror or a lens inside the irradiation optical system 33, and then passes through an acoustic mirror 42 transparent with respect to the light of these wavelengths. After that, the pulsed light converges to the inside of a brain 36 of the mouse. The Ti:sa laser 31 corresponds to the light source of an irradiator.

Photoacoustic waves generated from the brain 36 of the mouse are reflected by the acoustic mirror 42 and then received by a cMUT 35 serving as a capacitance type acoustic wave probe. The received signals are converted into electrical signals and then amplified. After that, the signals are stored in a memory 39 of a data acquiring system 37 as detection signals. When such measurement with the photoacoustic microscope is performed at the respective wavelengths, reception signals S₈₅₆(t), S₇₈₀(t), S₇₉₇(t), and S₈₂₅(t) are obtained. The cMUT 35 corresponds to a receptor.

In the embodiment, a thin film of a thickness of 30 nm serving as a standard sample is subjected in advance to photoacoustic measurement at the respective wavelengths. Thus, it is possible to acquire correction data IR(t) with consideration given to the impulse responses of the cMUT 35 and the influence of the pulse shapes of the respective wavelengths. The acquired correction data IR(t) is stored in the memory 39 in advance. In the embodiment, corrected reception signals are calculated using the correction data IR(t) according to the fifth correction method. A pulse shape difference corrector that performs the fifth correction method may be either the data acquiring system 37 or a workstation 38.

An oxygen saturation calculation mechanism 40 of the workstation 38 to which the calculated corrected reception signals are transferred calculates an oxygen saturation distribution and a hemoglobin concentration distribution from the corrected reception signals according to a program installed in advance. The calculated oxygen saturation distribution and the hemoglobin concentration distribution and their temporal changes are displayed on a monitor 41. The oxygen saturation calculation mechanism 40 corresponds to a spectral information acquirer.

(Processing Flow)

A description will be given of the spectral information acquiring method of the embodiment. The flow of the embodiment is basically the same as the flow of FIG. 4. In step S21, the brain 36 of the mouse is irradiated with the pulsed light 34 of wavelengths of 756 nm, 780 nm, 797 nm, and 825 nm. The cMUT 35 receives photoacoustic waves derived from the respective wavelengths and stores the reception signals in the memory 39 of the data acquiring system 37. In step S22, impulse responses are deconvolved from the reception signals of the respective wavelengths using the impulse responses with consideration given to the influence of the pulse shapes of beams of the pulsed light of the respective wavelengths. Thus, an inter-wavelength pulse shape difference is corrected, whereby it is possible to acquire corrected reception signals. In step S23, the oxygen saturation calculation mechanism 40 calculates an oxygen saturation distribution and a hemoglobin concentration distribution using the corrected reception signals at the respective wavelengths and causes the same to be displayed on the display 41.

According to the embodiment, in a photoacoustic microscope that uses light of a plurality of wavelengths, it is possible to correct a pulse width difference between wavelengths and accurately image a characteristic information distribution inside an object. The method of the embodiment is applicable to both a light focus type microscope having optical members for focusing light and an acoustic focus type microscope having acoustic members for focusing acoustic waves.

### Third Embodiment

A third embodiment will describe a time-resolved diffused light tomography apparatus. The apparatus measures a hemoglobin concentration distribution, the oxygen saturation distribution of hemoglobin, and the distribution of water or fat inside a human breast or the like.

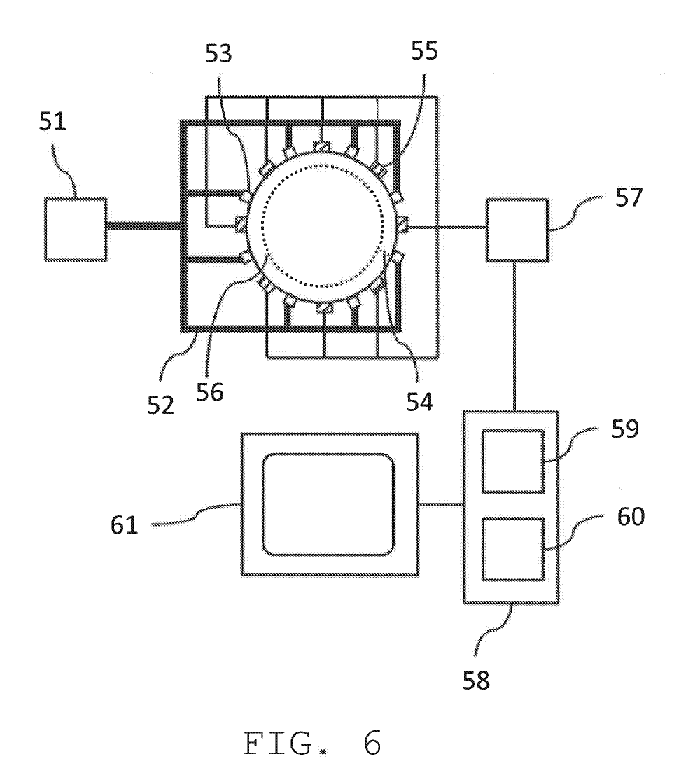

A description will be given of the configuration of the embodiment with reference to FIG. 6. A Ti:sa laser 51 is capable of successively emitting pulsed light of wavelengths of 750 nm to 850 nm. The wavelengths of the pulsed light preferably include but not limited to near-infrared regions. The emitted pulsed light passes through an optical fiber 52 and then is emitted from one of a plurality of emission ports 53 to a breast 56 as pulsed light 54. The pulsed light 54 is absorbed and scattered inside the breast 56. The pulsed light 54 propagates through breast tissues, and then is detected by a plurality of photomultiplier tubes 55. After being subjected to amplification processing, the reception signals are stored in the memory of a signal acquiring system 57. The Ti:sa laser 51 corresponds to the light source of an irradiator.

A pulse shape difference correction mechanism 59 of a PC 58 converts the reception signals inside the memory into corrected reception signals using the pulse shapes of the respective waves. It is preferable to directly couple the emission ports 53 and the photomultiplier tubes 55 to each other via optical fibers or the like to acquire the pulse shapes of the respective wavelengths and store the acquired pulse shapes in the memory of the PC in advance. A component concentration distribution calculation mechanism 60 calculates a moisture concentration distribution, a fat concentration distribution, a hemoglobin concentration distribution, and the oxygen saturation distribution of hemoglobin inside the breast 56 by an inverse problem analysis using the corrected reception signals. The component concentration distribution calculation mechanism 60 corresponds to a spectral information acquirer.

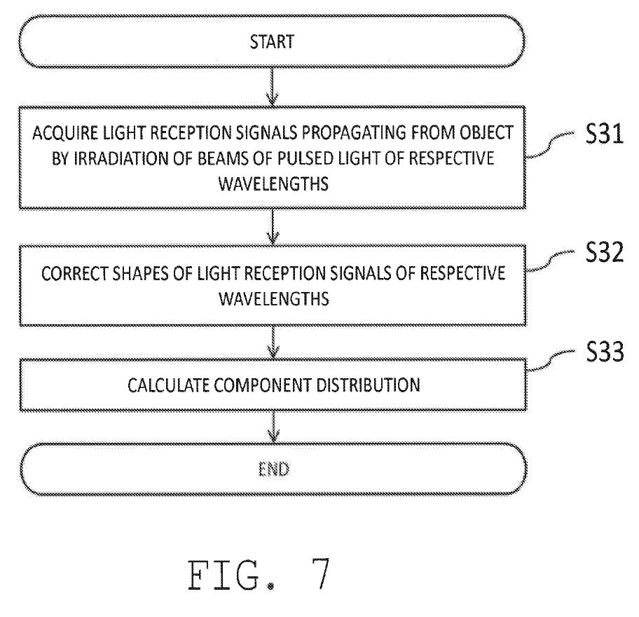

A description will be given of the spectral information acquiring method of the embodiment with reference to the flowchart of FIG. 7.

(Step S31)

Light reception signals obtained by the irradiation of beams of the pulsed light of respective wavelengths are acquired. That is, the breast 56 is irradiated with the pulsed light 54 of wavelengths of 750 nm to 850 nm in units of 1 nm. The photomultiplier tubes 55 serving as light detection elements store the reception signals in the data acquiring system 57.

(Step S32)

The light reception signals are corrected by the pulse shapes of the respective wavelengths. That is, the reception signals of the respective wavelengths are deconvolved using the previously-measured pulse shapes of the pulsed light of wavelengths of 750 nm to 850 nm. Thus, corrected reception signals may be acquired in which an inter-wavelength pulse shape difference is corrected.

(Step S33)

A component distribution is calculated. That is, a hemoglobin concentration distribution, the oxygen saturation distribution of hemoglobin, and the concentration distribution of water or fat inside an object are calculated by an inverse problem analysis using the corrected reception signals, and then displayed on the display 61.

According to the embodiment, in a spectral information acquiring apparatus that uses diffused light tomography based on a plurality of wavelengths, it is possible to correct a pulse shape difference between the wavelengths and accurately image a characteristic information distribution inside an object.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-15107, filed on Jan. 29, 2016, which is hereby incorporated by reference herein in its entirety.

What is claimed is:

1. An object information acquiring apparatus comprising:
an irradiator configured to emit pulsed light of a plurality of wavelengths different from each other;
a receiver configured to receive object signals propagating from the object irradiated with the pulsed light of the plurality of wavelengths and convert the received object signals into a plurality of reception signals;
a corrector configured to correct at least any one of the plurality of reception signals according to pulse shapes of the pulsed light of the plurality of wavelengths; and
an information acquirer configured to acquire spectral information on the object using the plurality of reception signals corrected by the corrector.

2. The object information acquiring apparatus according to claim 1, wherein
the receiver is configured to receive photoacoustic waves propagating from the object as the object signals.

3. The object information acquiring apparatus according to claim 1, wherein
the receiver is configured to receive as the object signals the light, which propagates after being absorbed and scattered in the object, since the light has been emitted from the irradiator.

4. The object information acquiring apparatus according to claim 1, wherein
the information acquirer is configured to acquire oxygen saturation of the object as the spectral information.

5. The object information acquiring apparatus according to claim 1, wherein
each of the pulse shapes indicates a time intensity change of the pulsed light.

6. The object information acquiring apparatus according to claim 1, wherein
when the pulse shapes of pulsed light of the plurality of wavelengths (1 to n) are indicated as P1(t) to Pn(t) and the plurality of reception signals are indicated as S1(t) to Sn(t), the corrector is configured to perform a correction so as to reduce influence on the plurality of reception signals, caused by a pulse shape difference between the pulsed light of a reference wavelength and the pulsed light of another wavelength.

7. The object information acquiring apparatus according to claim 6, wherein
the corrector is configured to perform the correction on the reception signal derived from the pulsed light of the other wavelength by performing calculation using the pulse shape of the pulsed light of the reference wavelength and the pulse shape of the pulsed light of the other wavelength.

8. The object information acquiring apparatus according to claim 6, wherein
the corrector is configured to use pulse widths of the pulsed light as the pulse shapes, and
the reference wavelength is a wavelength having a largest pulse width.

9. The object information acquiring apparatus according to claim 8, wherein
the corrector is configured to perform the correction on the reception signal derived from the pulsed light of the other wavelength such that a shape of the pulse width of the pulsed light of the other wavelength becomes same as a shape of the pulse width of the pulsed light of the reference wavelength.

10. The object information acquiring apparatus according to claim 9, wherein
the information acquirer is configured to acquire characteristic information on the object based on the reception signal corresponding to the pulsed light of the other wavelength and mask the spectral information using the characteristic information.

11. The object information acquiring apparatus according to claim 8, wherein
the corrector is configured to acquire the pulse widths by approximating the pulse shapes with a Gaussian function.

12. The object information acquiring apparatus according to claim 1, wherein
the corrector is configured to perform the correction by eliminating influence of the respective pulse shapes of the plurality of wavelengths from the plurality of reception signals.

13. The object information acquiring apparatus according to claim 1, wherein
the corrector is configured to acquire impulse responses of the receiver for the beams of pulsed light of the plurality of wavelengths, with the impulse responses being used for the correction.

14. The object information acquiring apparatus according to claim 1, wherein
the corrector is configured to perform the correction in a frequency domain.

15. The object information acquiring apparatus according to claim 1, wherein
the corrector is configured to perform the correction so as to reduce a pulse width difference between the plurality of reception signals caused by the pulse shapes of the beams of pulsed light of the plurality of wavelengths.

16. A signal processing method for a plurality of reception signals acquired by receiving object signals propagating from an object irradiated with pulsed light of a plurality of wavelengths different from each other,
the method comprising:
correcting at least any of the plurality of reception signals according to pulse shapes of beams of the pulsed light of the plurality of wavelengths; and
acquiring spectral information on the object using the plurality of reception signals corrected in the correction step.
